(19) United States
(12) Patent Application Publication
(10) Pub. No.: US 2004/0142970 A1
(43) Pub. Date: Jul. 22, 2004

(54) Treatment of hyperkinetic movement disorder with donepezil

(76) Inventors: Kathryn Chung, Lake Oswego, OR (US); Steven Johnson, Portland, OR (US)

Correspondence Address: DAVIS WRIGHT TREMAINE, LLP, 2600 CENTURY SQUARE, 1501 FOURTH AVENUE, SEATTLE, WA, 98101-1688, US

(21) Appl. No.: 10/698,963

(22) Filed: Oct. 31, 2003

Related U.S. Application Data
(60) Provisional application No. 60/422,930, filed on Nov. 1, 2002.

(51) Int. Cl. A61K 31/445

(52) U.S. Cl. 514/319

(57) ABSTRACT

This invention relates to methods and pharmaceutical compositions for treating hyperkinetic movement disorder, including dystonic tremor.

### CROSS REFERENCE TO RELATED APPLICATION

This Application claims the benefit of Provisional U.S. patent application Ser. No. 60/422,930, filed Nov. 1, 2002.

### FIELD OF THE INVENTION

The present invention concerns methods and pharmaceutical compositions for the treatment of hyperkinetic movement disorders including chorea and dystonic tremor.

### BACKGROUND OF THE INVENTION

Movement disorders can be classified into two basic categories: those characterized by disordered or excessive movement (referred to as “hyperkinesia” or “dyskinesia”), and those that are characterized by slowness, or a lack of movement (referred to as “hypokinesia,” “bradykinesia,” or “akinesia”). An example of a “hyperkinetic” movement disorder is a tremor or a tic while Parkinson's disease can be classified as “hypokinetic,” because it is often characterized by slow, deliberate movements, or even freezing in place.

Neurologic movement disorders include ataxia, corticobasal degeneration, dyskinesias (paroxysmal), dystonia (general, segmental, focal) including blepharospasm, spasmodic torticollis (cervical dystonia), writer's cramp (limb dystonia), laryngeal dystonia (spasmodic dysphonia), and oromandibular dystonia, essential tremor, hereditary spastic paraplegia, Huntington's Disease, multiple system atrophy (Shy Drager Syndrome), myoclonus, Parkinson's Disease, progressive supranuclear palsy, restless legs syndrome, Rett Syndrome, spasticity due to stroke, cerebral palsy, multiple sclerosis, spinal cord or brain injury, Sydenham's Chorea, tardive dyskinesia/dystonia, tics, Tourette's Syndrome, and Wilson's Disease.

Caroff, S. N. et al., J. Clin. Psychiatry 62:772-775 (2001) reported some response in a small group of patients with tardive dyskinesia treated with donepezil. However, Tammenmaa, I. A. et al., Cochrane Database Syst. Rev. 2000:CD000207 (2002) reviewed the effects of a number of drugs, including donepezil, on treatment of tardive dyskinesia and concluded that there was no substantial improvement in tardive dyskinesia symptoms when compared with placebo. Due to these results, and to the side effects of many current treatments for hyperkinetic movement disorders, there is a need in the art for improved therapies.

### SUMMARY OF THE INVENTION

The present invention provides, by one of its aspects, a pharmaceutical composition for the amelioration of hyperkinetic movement disorders, comprising as an active ingredient, a pharmaceutically effective amount of a cholinesterase inhibitor, such as donepezil.

The present invention provides, by another of its aspects, use of donepezil for the preparation of a pharmaceutical composition for the amelioration of hyperkinetic movement disorders.

In a preferred embodiment, the pharmaceutical composition comprises about 1 mg to about 50 mg of donepezil.

In a more preferred embodiment, the pharmaceutical composition comprises about 2 mg to about 25 mg of donepezil.

The invention further provides a method for ameliorating hyperkinetic movement disorder, comprising administering to a subject in need of such treatment a therapeutically effective amount of donepezil.

In one embodiment, the method comprises administering about 1 mg to about 50 mg of donepezil to the subject per day.

In a further embodiment, the method comprises administering about 2 mg to about 25 mg of donepezil to the subject per day.

The dosage of the active ingredient should be tested empirically for each specific indication, and depends on various factors, such as the patient's weight, the length of time of administration of the donepezil, age, etc. Generally speaking, the dosage should be of about 1 to about 50 mg per day, preferably of about 2 to about 25 mg per day, most preferably of about 2.5 to about 10 mg per day.

The pharmaceutical composition of the invention may comprise donepezil and a pharmaceutically acceptable carrier.

### DETAILED DESCRIPTION OF THE INVENTION

The invention relates in one aspect to treatment of dystonia, which is a neurologic movement disorder characterized by sustained muscle contractions, usually producing twisting and repetitive movements or abnormal postures or positions. Almost all dystonic movements share a directional quality that is typically sustained, sometimes for an instant, as well as a consistency and predictability. Dystonia movements are directional, forcing the involved body part or region into an abnormal position, which is consistently present.

Dystonia can be classified by age at which symptoms appear. Symptoms may become apparent during childhood, adolescence, or adulthood. It can also classified by the area or areas of the body that are affected. Sustained muscle contractions and abnormal movement patterns may be limited to one area of the body; involve two or more areas of the body that are next to each other, as in segmental dystonia; or two or more areas of the body that are not next to each other (non segmental or multi focal); or be generalized in nature, including leg involvement and other areas of the body. Dystonia can also be classified by cause. It may occur as a primary condition (idiopathic dystonia) that is familial or occurs in the absence of a family history. It may result from certain environmental factors or “insults” that affect the brain (secondary or symptomatic dystonia). Dystonia may be associated with certain nondegenerative, neurochemical disorders (known as “dystonia plus syndromes”) that are characterized by neurologic features, such as parkinsonism or myoclonus. Dystonia is also a primary feature of certain, usually hereditary, neurodegenerative disorders (so called “heredodegenerative dystonias”).

Currently, there are a number of treatment options available to treat dystonia, but all have side effects or other disadvantages. Drugs may be used alone or in combination. In addition, they may be combined with other forms of treatment. Dystonia can be accompanied by dystonic tremor.

Botulinum toxin (BTX) is a biological therapeutic agent that can be effective in treating dystonia. Botulinum toxin is a toxic protein produced by the bacterium Clostridium botulinum. BTX can cause botulism, a severe form of food poisoning that is contracted through the ingestion of contaminated food products. However, when a minute amount of commercially prepared BTX is injected directly into an overactive muscle, it relaxes the muscle, by blocking the release of acetylcholine, a neurotransmitter responsible for activation of muscle contraction. Thus, BTX decreases inappropriate or excessive muscle contractions, allowing the affected area (e.g., arm, neck, leg, eyelid, etc.) to assume a more normal position or posture.

Benzodiazepines are a class of drugs that interfere with chemical activities in the nervous system and brain, serving to reduce communication between nerve cells. Such medications may relax muscles and ease symptoms associated with dystonia. Benzodiazepines are oral medications that may be used to treat focal, segmental, and generalized dystonias. Diazepam (Valium®) and clonazepam (Klonopin®) are two types of benzodiazepines most commonly used to treat dystonia. The major side effect of these drugs is drowsiness, which may be controlled by lowering the dose. At relatively high doses, side effects may include depression, personality changes, or, in severe cases, psychosis.

Baclofen (Lioresal®) is used to treat individuals with spasticity, but it has also been administered to some patients with dystonia. Baclofen's primary site of action is the spinal cord where it reduces the release of neurotransmitters that stimulate muscle activity (GABA agonist stimulating GABAB autoreceptor). Baclofen has been used to treat both primary and secondary dystonias, and may be administered orally or through a surgically implanted pump that delivers the drug directly to the spinal cord (intrathecal baclofen).

Anticholinergic drugs block the action of the neurotransmitter acetylcholine, thereby deactivating muscle contractions. These drugs are administered orally and used to treat focal, segmental, and generalized dystonias. Trihexyphenidyl (Artane®) and diphenhydramine (Benadryl®) are the most common anticholinergic agents used to treat dystonia. This form of therapy may be more beneficial in children, as they may be able to tolerate higher doses of trihexphenidyl than adults. Greater therapeutic benefits may also occur in patients who initiate drug therapy early during the course of their disease. Side effects may be severe, particularly at higher doses, and may include confusion, drowsiness, hallucinations, forgetfulness, personality changes, dry mouth, blurred vision, and urinary retention.

Dopamine blocking or dopamine depleting agents may be used to treat some patients with dystonia. The possible positive effect of these agents is a paradox since dopamine blockers may also cause dystonia. Nonetheless, these agents have been shown to be effective in some patients. Although not currently available in the United States, tetrabenazine is the most widely used dopamine blocking agent. In some patients, tetrabenazine may be combined with lithium, which may help to lessen side effects such as slowed movements and depression. Other dopamine blockers are not as commonly used, since they may be more likely to evoke tardive dystonia. The neuroleptic drugs clozapine and olanzapine may be useful for the treatment of dystonia and may be less likely to cause tardive dystonia.

The acetylcholesterase inhibitor donepezil HCl, also known as Aricept®, has been used to treat patients with Alzheimer's Disease. However, donepezil has not been shown to be effective in other conditions such as progressive supranuclear palsy (Litvan, I. et al., Neurology 57:467-473, 2001), or Parkinson's Disease with dementia, according to a report by Aarsland, D. et al. as reported at www.wemove.org in 2001. Donepezil is also known chemically as (±)-2,3-dihydro-5,6-dimethoxy-2-[[1-(phenylmethyl)-4-piperidinyl]methyl]-1H-inden-1-one hydrochloride, and also as E2020.

In addition to treatment of dystonia with donepezil, the invention also relates to use of donepezil for treatment of other hyperkinetic movement disorders. The term “hyperkinetic movement disorders” refers to conditions such as tremor, chorea, tics, dyskinesia, and dystonia, including dystonic tremor. Hyperkinetic movement disorders can be classified generally into several catogories, including tics, tremors, dyskinesia, and chorea. Tremors can be classified as essential and dystonic, with essential being the more common of the two. Dyskinesias can be idiopathic and tardive.

The following definitions apply herein:

The term “amelioration” refers to a decrease in the abnormal involuntary movements characterizing hyperkinetic movement disorders, as can be determined for example, by using the Abnormal Involuntary Movement Scale (AIMS).

The term “effective amount” refers to an amount that brings about a reduction in the AIMS.

Essential tremor (ET) is a common, slowly and variably progressive neurologic movement disorder characterized by involuntary, rhythmic, “back and forth” movements (i.e., tremor) of a body part or parts. In ET patients, tremor is primarily a “postural” or “kinetic” tremor or may be a combination of both types: i.e., tremor occurs while voluntarily maintaining a fixed position against gravity (postural tremor) and/or when conducting self directed, targeted actions (kinetic intention tremor). In many individuals with ET, both hands are affected, although the condition may sometimes initially be noted in the dominant hand. ET also frequently affects the head, with tremor occurring in a horizontal pattern in most patients and the remainder affected by vertical tremors. Less commonly, patients have tremor involving the voice, tongue, or roof of the mouth (palate), leading to impaired articulation of speech (dysarthria). Rarely, tremor may affect the trunk or lower limbs, particularly with advanced stages of disease. ET may appear to occur randomly for unknown reasons (sporadically) or be transmitted as an autosomal dominant trait.

Huntington's disease (HD) is a hereditary, progressive, neurodegenerative disorder primarily characterized by the development of emotional, behavioral, and psychiatric abnormalities; gradual deterioration of thought processing and acquired intellectual abilities (dementia); and movement abnormalities, including involuntary, rapid, irregular jerky movements (chorea) of the face, arms, legs, or trunk. HD may be inherited as an autosomal dominant trait or, less commonly, appear to occur randomly for unknown reasons (sporadically). The disorder results from abnormally long sequences or “repeats” of certain coded instructions (i.e., unstable expanded CAG repeats) within a gene (located on chromosome p16.3). Progressive nervous system dysfunction associated with HD results from loss of neurons in certain areas of the brain, including the basal ganglia and cerebral cortex.

Parkinson's disease (PD) is a slowly progressive degenerative disorder of the central nervous system characterized by slowness or lack of movement (bradykinesia), rigidity, postural instability, and tremor primarily while at rest. Additional characteristics include a shuffling, unbalanced manner of walking; forward bending or flexion of the trunk; a fixed or “mask like” facial expression; weakness of the voice; abnormally small, cramped handwriting (micrographia); depression; or other symptoms and findings. Such abnormalities may result from progressive loss of nerve cells within a certain region of the substantia nigra of the brain and the associated depletion of the neurotransmitter dopamine.

Tardive dyskinesia is a movement disorder that may result from extended therapy with certain antipsychotic medications such as haloperidol. The condition is characterized by involuntary, rhythmic movements of the face, jaw, mouth, and tongue, such as lip pursing, chewing movements, or protrusion of the tongue. Facial movements are sometimes accompanied by involuntary, jerky or writhing motions (choreoathetoid movements) of the trunk, arms, and legs. In some patients, symptoms discontinue months or years after withdrawal of antipsychotic therapy. However, in others, the condition may not be reversible.

Tardive dystonia is a form of dyskinesia characterized by chronic dystonia due to administration of medications that block dopamine D2 receptors (dopamine receptor antagonists), such as certain antipsychotic agents. (Dopamine receptors are molecules on the surfaces of receiving nerve cells that are sensitive to stimulation by dopamine, a neurotransmitter that controls movement and balance. Several types of dopamine receptors have been identified, including DI, D2, and D3.) Dystonia is a neurologic movement disorder characterized by sustained muscle contractions that often result in repetitive twisting motions or unusual postures or positions. Tardive dystonia is the most common form of secondary dystonia, specifically, dystonia that results from certain environmental factors or “insults” that affect the brain. In adults, tardive dystonia often initially affects facial or neck muscles. Dystonia may remain limited to such regions or extend to affect adjacent muscles of the trunk and arms. Children are more likely to be affected by generalized dystonia that involves muscles of the trunk and legs.

Tics are defined as involuntary, compulsive, stereotypic muscle movements or vocalizations that abruptly interrupt normal motor activities. These repetitive, purposeless motions (motor tics) or utterances (vocal tics) may be simple or complex in nature; may be temporarily suppressed; and are often preceded by a “foreboding” sensation or urge that is temporarily relieved following their execution. Simple tics include abrupt, isolated movements, such as repeated facial twitching, blinking, or shoulder shrugging, and simple sounds, including grunting, throat clearing, or sighing. Complex tics may involve more sustained, complex movements, such as deep knee bending or leg kicking, or complex vocalizations, including repeating another person's words or phrases (echolalia) or, rarely, explosive cursing (coprolalia). Tourette syndrome is defined as the presence of multiple motor and vocal tics for at least one year, changes in the nature of the tics (e.g., complexity, severity, anatomical location) during the course of the disorder, and symptom onset before age 21.

The invention will now be described with reference to the following non-limiting example.

### EXAMPLE

Treatment of Patients with Hyperkinetic Movement Disorders

The centrally active cholesterase inhibitor donepezil was used to treat a variety of patients with hyperkinetic movement disorders. The diagnoses included idiopathic chorea, idiopathic generalized dystonia, essential tremor, dystonic tremor, and oromandibular dyskinesia. Results from patients showing improvement are shown in Table 1. Response was judged by clinical exam and history.

TABLE 1

| | | | | Dose | Treatment | |
|---|---|---|---|---|---|---|
| | Age | | | Regimen | Duration | |
| Patient | (y) | Sex | Diagnosis | (mg) | (wks) | Response |
| 1 | 32 | F | Tremor with | 5 + 2.5 | 26 | Marked improvement |
| | | | subtle dystonia | | | |
| 2 | 45 | F | Tremor with | 5 + 5 | 12 | Marked improvement |
| | | | dystonia | | | |
| 3 | 38 | M | Tremor with | 5 + 5 | 20 | Marked improvement |
| | | | dystonia | | | |
| 4 | 72 | M | Action tremor | 5 + 5 | 12 | Marked improvement |
| 5 | 52 | F | Tremor with | 5 + 5 | 12 | Moderate |
| | | | dystonia | | | improvement |
| 6 | 35 | F | Tremor with | 5 + 5 | 6 | Moderate to marked |
| | | | dystonia | | | improvement |
| 7 | 43 | F | Isolated head | 5 | 6 | Marked improvement |
| | | | tremor | | | |
| 8 | 17 | M | Essential | 5 | 4 | Marked improvement |
| | | | tremor vs | | | |
| | | | dystonic | | | |
| | | | tremor | | | |
| 9 | 63 | M | Idiopatic | 5 + 10 | 28 | Moderate |
| | | | Chorea | | | improvement |
| 10 | 82 | F | Idiopathic | 5 + 5 | 136 | Marked |
| | | | Chorea | | | improvement, |
| | | | | | | lessened effect after |
| | | | | | | 2 years |
| 11 | 70 | F | Idiopathic | 5 + 7.5 | 2 | Moderate |
| | | | chorea | | | improvement |
| 12 | 71 | F | APLA assoc | 5 + 5 | 10 | Moderate |
| | | | chorea | | | improvement |
| 13 | 79 | M | Chorea | 10 | 22 | Moderate |
| | | | idiopathic | | | improvement |
| 14 | 78 | M | Chorea | 10 | 20 | Mild to moderate |
| | | | idiopathic, | | | improvement |
| | | | familial | | | |
| 15 | 45 | F | Idiopathic | 5 + 5 + 5 | 28 | Marked improvement |
| | | | generalized | | | |
| | | | dystonia | | | |
| 16 | 58 | F | Torticollis | 5 | 2 | Resolution of |
| | | | | | | dystonic shoulder |
| | | | | | | pain and a |
| | | | | | | swallowing |
| | | | | | | difficulty. |
| 17 | 32 | M | Tourette's | 5 + 5 | 24 | Marked improvement |
| | | | syndrome | | | in motor tics, less in |
| | | | | | | vocal |
| 18 | 48 | M | Tourette's | 5 + 5 | 20 | Marked improvement |
| | | | syndrome | | | in motor/vocal tics |
| 19 | 58 | F | Tic disorder | 5 + 2.5 | | Moderate |
| | | | | | | improvement |
| 20 | 59 | M | Orthostatic | 5 | 8 | Moderate |
| | | | tremor | | | improvement |

The results indicate that raising brain acetylcholine may ameliorate a variety of hyperkinetic movement disorders. The most impressive result has been achieved in patients with tremor and concomitant dystonia, where markedly positive results were consistently found. While donepezil has a plasma half life of 70 hours, some patients have required dosing usually twice a day, to control symptoms. These open label results suggest that acetylcholinesterase inhibitors are useful for treating hyperkinetic movement disorders, particularly chorea and dystonia.

The foregoing specification, including the specific embodiments and examples, is intended to be illustrative of the present invention and is not to be taken as limiting. Numerous other variations and modifications can be effected without departing from the true spirit and scope of the invention. All patents, patent publications, and non-patent publications cited are incorporated by reference herein.

What is claimed is:

1. A pharmaceutical composition for the treatment of hyperkinetic movement disorder comprising an effective amount of a cholinesterase inhibitor and a pharmaceutically acceptable carrier.

2. A pharmaceutical composition according to claim 1 wherein said cholinesterase inhibitor is donepezil.

3. A pharmaceutical composition according to claim 1, comprising about 1 to about 50 mg of donepezil.

4. A method for ameliorating hyperkinetic movement disorder comprising administering to a subject in need of such treatment, a therapeutically effective amount of donepezil.

5. A method according to claim 4, wherein the donepezil is administered in an amount of about 1 to about 50 mg per day.

6. A method according to claim 5, wherein the donepezil is administered in an amount of about 2.5 to 25 mg per day.

7. A method according to claim 6, wherein the donepezil is administered in an amount of about 2 to 10 mg per day.

8. A method for the treatment of dystonic tremor comprising administering to a subject in need of such treatment a cholinesterase inhibitor.

9. A method according to claim 8, wherein said cholinesterase inhibitor is donepezil.

10. A method according to claim 9, wherein the donepezil is administered in an amount of about 1 to about 50 mg per day.
